(19) United States
(12) Patent Application Publication
(10) Pub. No.: US 2016/0129080 A1
(43) Pub. Date: May 12, 2016

(54) TREATMENT OF POLYPOIDAL CHROIDAL VASCULOPATHY

(71) Applicant: Aaron OSBORNE, Fort Worth, TX (US)

(72) Inventor: Aaron OSBORNE, Fort Worth, TX (US)

(21) Appl. No.: 14/898,264

(22) PCT Filed: Jun. 18, 2014
(86) PCT No.: PCT/IB2014/062369

§ 371 Date: Dec. 14, 2015

Related U.S. Application Data
(60) Provisional application No. 61/837,247, filed on Jun. 20, 2013.

(51) Int. Cl. A61K 38/17; A61K 9/00; A61K 41/00

(52) U.S. Cl. CPC A61K 38/179; A61K 41/0071; A61K 9/0048; A61K 9/0019

(57) ABSTRACT

Patients with polypoidal choroidal vasculopathy receive a combination of both (i) a photodynamic therapy, such as verteporfin and (ii) a non-antibody VEGF antagonist.

### TECHNICAL FIELD

This invention is in the field of treating polypoidal choroidal vasculopathy.

### BACKGROUND ART

Polypoidal choroidal vasculopathy (PCV) was first described as a peculiar hemorrhagic disorder of the macula, characterized by recurrent sub-retinal and sub-retinal pigment epithelium bleeding in middle aged black women. The primary abnormality involves the choroidal circulation, and the characteristic lesion is an inner choroidal vascular network of vessels ending in an aneurysmal bulge or outward projection, visible clinically as a reddish orange, spheroid, polyp-like structure. The presence of hyperfluorescent nodules that appear in the early phase (first five minutes) of fundus indocyanine green angiography (ICGA) is a diagnostic hallmark of this disease. See Lim et al. (2010) Eye 24:483-490. For the diagnosis of PCV, at least one of the following criteria must additionally be fulfilled: (1) presence of hypofluorescent halo surrounding the lesion, (2) presence of pulsation, (3) association with a branching vascular network, (4) correspondence to an orange-red nodule in fundus photographs, (5) a nodular appearance (rather than a flat lesion) when viewed in stereo pairs, or (6) associated with massive haemorrhage. People of African-American and Asian descent are more at risk for developing PCV as the disorder seems to preferentially affect pigmented individuals. The natural course of the disease often follows a remitting-relapsing course, and clinically, it is associated with chronic, multiple, recurrent serosanguineous detachments of the retinal pigment epithelium and neurosensory retina with long-term preservation of good vision. See Yuzawa et al. (2005) Br J Ophthalmol 89:602-7.

Standard treatment for PCV has involved verteporfin (Visudyne™), which is a photodynamic (light-activated) drug that is injected into the bloodstream. The drug travels to the abnormal vessels in the eye where it is activated by a low-energy non-thermal laser to produce a reaction which damages and closes the vessels. See Spaide et al. (2002) Retina 22:529-35.

It is an object of the invention to provide further and improved treatments for PCV.

### DISCLOSURE OF THE INVENTION

Photodynamic verteporfin treatment is effective on its own, but improvements could be achieved by using it in combination with other treatments. According to the invention, patients with PCV receive a combination of both (i) a photodynamic therapy, such as verteporfin and (ii) a non-antibody VEGF antagonist. This combination therapy can provide better results than either treatment alone, and advantageously can provide a synergistic improvement. Usefully, the downstream efficacy of the therapy can be improved e.g. a greater proportion of patients will have complete polyp regression 6 months after the combination therapy than 6 months after either of the two individual therapies. Photodynamic therapy increases the efficacy of the treatment with a non-antibody VEGF antagonist.

Without being bound by any particular theory, the inventors believe that the photodynamic therapy treats the root cause of PCV by inducing polyp regression, whereas the non-antibody VEGF antagonist therapy reduces vascular permeability and dries up the macular edema in the affected eye thereby improving the patient's vision.

The invention therefore provides a non-antibody VEGF antagonist for use in a method for treating a patient having PCV, wherein said method comprises administering to the patient a combination of both (i) a photodynamic therapy and (ii) a non-antibody VEGF antagonist. The non-antibody VEGF antagonist is typically administered intravitreally.

The invention further provides the use of a non-antibody VEGF antagonist in the manufacture of a medicament for treating a patient having PCV, wherein the non-antibody VEGF antagonist is for administration in combination with a photodynamic therapy.

### Photodynamic Therapy

The invention involves administration of a photodynamic therapy. The photodynamic therapy will typically involve administration of a photosensitiser remote from the eye, by injection, from where it is transported (e.g. by binding to lipoproteins in plasma) to the eye. The photosensitiser is activated locally by illumination of defined area of the vasculature via a laser beam. Light activation causes the photosensitiser to become highly reactive resulting in damage to the vasculature. Damaged endothelium is known to release procoagulant and vasoactive factors through the lipooxygenase (leukotriene) and cyclooxygenase (eicosanoids such as thromboxane) pathways, resulting in platelet aggregation, fibrin clot formation and vasoconstriction, closing off blood vessels in the area of photo activation.

The most preferred photosensitiser is verteporfin. In the presence of oxygen, light-activated verteporfin at the back of the eye forms highly reactive short-lived singlet oxygen and reactive oxygen radicals which cause local damage to neovascular endothelium, resulting in the desired therapeutic effect. Verteporfin is typically administered by intravenous infusion (e.g. over a period of 10 minutes) at a dose of 6 mg/m² body surface area. Verteporfin can be supplied in lyophilised form (including excipients such as lactose monohydrate, egg phosphatidylglycerol, dimyristoyl phosphatidylcholine, ascorbyl palmitate and butylated hydroxytoluene) which can be reconstituted into 7 ml water for infusion to provide a 2 mg/ml concentrated solution in 7.5 ml. The concentrated solution can then be diluted in a 50 mg/ml glucose solution (verteporfin precipitates in sodium chloride solution) to give a final desired volume e.g. to give a final volume of 30 ml, with 0.5 mg/ml verteporfin. This final solution can be delivered via an infusion line, filtered using a hydrophilic membrane with a pore size of not less than 1.2 μm. The next step with verteporfin is light activation, which can commence 15 minutes after the start of infusion. A diode laser generating non-thermal red light (689±3 nm) can be used via a slit lamp mounted fibre optic device and a suitable contact lens. At a light intensity of 600 mW/cm² it takes 83 seconds to deliver a light dose of 50 J/cm². Depending on the desired treatment effect or the size of the area affected by PCV, the light intensity or duration of the exposure may be varied. For example, during half-fluence PDT with verteporfin, the delivered laser energy is reduced to half of the standard dose, i.e. 25 J/cm² over 83 seconds. Since less light energy is delivered, less photosensitiser is activated resulting in less damage, in particular to the area directly surrounding the endothelium, and potentially reducing adverse side effects. As an alternative to reducing the light intensity, the duration of the light exposure may be reduced. For example, the exposure time may be reduced by 25%, 50% or 75%.

If a patient requires treatment of both eyes then these are typically not treated together. Instead, light should be applied to the second eye immediately after light application in the first eye.

### Non-Antibody VEGF Antagonists

VEGF is a well-characterised signal protein which stimulates angiogenesis. Two anti-VEGF antibody antagonists have been approved for human use, namely ranibizumab (Lucentis™) and bevacizumab (Avastin™).

In one aspect of the invention, the non-antibody VEGF antagonist is an immunoadhesin. One such immuoadhesin is aflibercept (Eylea®), which has recently been approved for human use and is also known as VEGF-trap (Holash et al. (2002) PNAS USA 99:11393-98; Riely & Miller (2007) Clin Cancer Res 13:4623-7s). Aflibercept is the preferred non-antibody VEGF antagonist for use with the invention. Aflibercept is a recombinant human soluble VEGF receptor fusion protein consisting of portions of human VEGF receptors 1 and 2 extracellular domains fused to the Fc portion of human

IgG1. It is a dimeric glycoprotein with a protein molecular weight of 97 kilodaltons (kDa) and contains glycosylation, constituting an additional 15% of the total molecular mass, resulting in a total molecular weight of 115 kDa. It is conveniently produced as a glycoprotein by expression in recombinant CHO K1 cells. Each monomer can have the following amino acid sequence (SEQ ID NO: 1):

SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDWLSPSHGIELSVGEKLVLNCTARTELNVGIDFNWEYPSSKHQHKKLV
NRDLKTQSGSEMKKFLSTLTIDGVTRSDQGLYTCAASSGLMTKKNSTEVR
VHEKDKTHTCPPCPAPELLGGPSVFLFPPKPKDTLMISRTPEVTCVWDVS
HEDPEVKFNWYVDGVEVHNAKTKPREEQYNSTYRWSVLTVLHQDWLNGKE
YKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQ
QGNVFSCSVMHEALHNHYTQKSLSLSPG

and disulfide bridges can be formed between residues 30-79, 124-185, 246-306 and 352-410 within each monomer, and between residues 211-211 and 214-214 between the monomers.

Another non-antibody VEGF antagonist immunoadhesin currently in pre-clinical development is a recombinant human soluble VEGF receptor fusion protein similar to VEGF-trap containing extracellular ligand-binding domains 3 and 4 from VEGFR2/KDR and domain 2 from VEGFR1/Flt-1; these domains are fused to a human IgG Fc protein fragment (Li et al., 2011 Molecular Vision 17:797-803). This antagonist binds to isoforms VEGF-A, VEGF-B and VEGF-C. The molecule is prepared using two different production processes resulting in different glycosylation patterns on the final proteins. The two glycoforms are referred to as KH902 (conbercept) and KH906. The fusion protein can have the following amino acid sequence (SEQ ID NO:2):

MVSYWDTGVLLCALLSCLLLTGSSSGGRPFVEMYSEIPEIIKMTEGREL
VIPCRVTSPNITVTLKKFPLDTLIPDGKRIIWDSRKGFIISNATYKEIG
LLTCEATVNGHLYKTNYLTHRQTNTIIDVVLSPSHGIELSVGEKLVLNC
TARTELNVGIDFNWEYPSSKHQHKKLVNRDLKTQSGSEMKKFLSTLTID
GVTRSDQGLYTCAASSGLMTKKNSTFVRVHEKPFVAFGSGMESLVEATV
GERVRLPAKYLGYPPPEIKWYKNGIPLESNHTIKAGHVLTIMEVSERDT
GNYTVILTNPISKEKQSHVVSLVVYVPPGPGDKTHTCPLCPAPELLGGP
SVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNG
QPENNYKATPPVLDSDGSFFLYSKLTVDKSRWQQGNVFSCSVMHEALHN
HYTQKSLSLSPGK

and, like VEGF-trap, can be present as a dimer This fusion protein and related molecules are further characterized in EP1767546.

Other non-antibody VEGF antagonists include antibody mimetics (e.g. Affibody® molecules, affilins, affitins, anticalins, avimers, Kunitz domain peptides, and monobodies) with VEGF antagonist activity. This includes recombinant binding proteins comprising an ankyrin repeat domain that binds VEGF-A and prevents it from binding to VEGFR-2. One example for such a molecule is DARPin® MP0112. The ankyrin binding domain may have the following amino acid sequence (SEQ ID NO: 3):

GSDLGKKLLEAARAGQDDEVRILMANGADVNTADSTGWTPLHLAVPWGH
LEIVEVLLKYGADVNAKDFQGWTPLHLAAAIGHQEIVEVLLKNGADVNA
QDKFGKTAFDISIDNGNEDLAEILQKAA

Recombinant binding proteins comprising an ankyrin repeat domain that binds VEGF-A and prevents it from binding to VEGFR-2 are described in more detail in WO2010/060748 and WO2011/135067.

Further specific antibody mimetics with VEGF antagonist activity are the 40 kD pegylated anticalin PRS-050 and the monobody angiocept (CT-322).

The non-antibody VEGF antagonist may be modified to further improve their pharmacokinetic properties or bioavailability. For example, a non-antibody VEGF antagonist may be chemically modified (e.g., pegylated) to extend its in vivo half-life. Alternatively or in addition, it may be modified by glycosylation or the addition of further glycosylation sites not present in the protein sequence of the natural protein from which the VEGF antagonist was derived.

Variants of the above-specified VEGF antagonists that have improved characteristics for the desired application may be produced by the addition or deletion of amino acids. Ordinarily, these amino acid sequence variants will have an amino acid sequence having at least 60% amino acid sequence identity with the amino acid sequences of SEQ ID NO: 1, SEQ ID NO: 2 or SEQ ID NO: 3, preferably at least 80%, more preferably at least 85%, more preferably at least 90%, and most preferably at least 95%, including for example, 80%, 81%, 82%, 83%, 84%, 85%, 86%, 87%, 88%, 89%, 90%, 91%, 92%, 93%, 94%, 95%, 96%, 97%, 98%, 99%, and 100%. Identity or homology with respect to this sequence is defined herein as the percentage of amino acid residues in the candidate sequence that are identical with SEQ ID NO: 1, SEQ ID NO: 2 or SEQ ID NO: 3, after aligning the sequences and introducing gaps, if necessary, to achieve the maximum percent sequence identity, and not considering any conservative substitutions as part of the sequence identity.

Sequence identity can be determined by standard methods that are commonly used to compare the similarity in position of the amino acids of two polypeptides. Using a computer program such as BLAST or FASTA, two polypeptides are aligned for optimal matching of their respective amino acids (either along the full length of one or both sequences or along a pre-determined portion of one or both sequences). The programs provide a default opening penalty and a default gap penalty, and a scoring matrix such as PAM 250 [a standard scoring matrix; see Dayhoff et al., in Atlas of Protein Sequence and Structure, vol. 5, supp. 3 (1978)] can be used in conjunction with the computer program. For example, the percent identity can then be calculated as: the total number of identical matches multiplied by 100 and then divided by the sum of the length of the longer sequence within the matched span and the number of gaps introduced into the longer sequences in order to align the two sequences.

Non-antibody VEGF antagonists are preferred herein over antibody VEGF antagonists due their different pharmacokinetic profile when administered intravitreally. Preferably, the non-antibody

VEGF antagonist of the invention binds to VEGF via one or more protein domain(s) that are not derived from the antigen-binding domain of an antibody. The non-antibody VEGF antagonist of the invention are preferably proteinaceous, but may include modifications that are non-proteinaceous (e.g., pegylation, glycosylation).

### Administration

The non-antibody VEGF antagonist of the invention will generally be administered to the patient via intravitreal injection, though other routes of administration may be used, such as a slow-release depot, an ocular plug/reservoir or eye drops. Administration in aqueous form is usual, with a typical volume of 20-150 μl e.g. 40-60 μl, or 50 μl. Injection can be via a 30-gauge×½-inch (12.7 mm) needle. For example, aflibercept is generally administered via intravitreal injection at a dose of 2 mg (suspended in 0.05 mL buffer comprising 40 mg/mL in 10 mM sodium phosphate, 40 mM sodium chloride, 0.03% polysorbate 20, and 5% sucrose, pH 6.2). However, the normal dose may be reduced for the treatment of smaller children and in particular infants. The dose for treating an infant with a VEGF antagonist of the invention is usually 50% of the dose administered to an adult. Smaller doses (e.g., 0.5 mg per monthly injection) may also be used.

Alternatively, an intravitreal device is used to continuously deliver a non-antibody VEGF antagonist into the eye over a period of several months before needing to be refilled by injection. Various intravitreal delivery systems are known in the art. These delivery systems may be active or passive. For example, WO2010/088548 describes a delivery system having a rigid body using passive diffusion to deliver a therapeutic agent. WO2002/100318 discloses a delivery system having a flexible body that allows active administration via a pressure differential. Alternatively, active delivery can be achieved by implantable miniature pumps. An example for an intravitreal delivery system using a miniature pump to deliver a therapeutic agent is the Ophthalmic MicroPump System™ marketed by Replenish, Inc. which can be programmed to deliver a set amount of a therapeutic agent for a pre-determined number of times.

The non-antibody VEGF antagonist is typically encased in a small capsule-like container (e.g., a silicone elastomer cup).The container is usually implanted in the eye above the iris. The container comprises a release opening. Release of the non-antibody VEGF antagonist may be controlled by a membrane positioned between the non-antibody VEGF antagonist and the opening, or by means of a miniature pump connected to the container. Alternatively, the non-antibody VEGF antagonist may be deposited in a slow-release matrix that prevents rapid diffusion of the antagonist out of the container.

Preferably, the intravitreal device is designed to release the non-antibody VEGF antagonist at an initial rate that is higher in the first month. The release rate slowly decreases, e.g., over the course of the first month after implantation, to a rate that is about 50% less than the initial rate. The container may have a size that is sufficient to hold a supply of the non-antibody VEGF antagonist that lasts for about four to six months. Since a reduced dose of VEGF antagonist may be sufficient for effective treatment when administration is continuous, the supply in the container may last for one year or longer, preferably about two years, more preferably about three years.

Continuous delivery of the VEGF antagonist may be particularly advantageous in patients that require continuous treatment with a VEGF antagonist to halt or slow progressive loss of visual acuity. Because only a small surgery is required to implant a delivery system and intravitreal injections are avoided, patient compliance issues can be avoided.

In one aspect of the invention, the non-antibody VEGF antagonist is provided in a pre-filled sterile syringe ready for administration. Preferably, the syringe has low silicone content. More preferably, the syringe is silicone free. The syringe may be made of glass. Using a pre-filled syringe for delivery has the advantage that any contamination of the sterile antagonist solution prior to administration can be avoided. Pre-filled syringes also provide easier handling for the administering ophthalmologist.

### Slow-Release Formulations

Non-antibody VEGF antagonist may be provided as slow-release formulations. Slow-release formulations are typically obtained by mixing a therapeutic agent with a biodegradable polymer or encapsulating it into microparticles. By varying the manufacture conditions of polymer-based delivery compositions, the release kinetic properties of the resulting compositions can be modulated.

A slow-release formulation in accordance with the invention typically comprises a non-antibody VEGF antagonist, a polymeric carrier, and a release modifier for modifying a release rate of the non-antibody VEGF antagonist from the polymeric carrier. The polymeric carrier usually comprises one or more biodegradable polymers or co-polymers or combinations thereof. For example, the polymeric carrier may be selected from poly-lactic acid (PLA), poly-glycolic acid (PGA), poly-lactide-co-glycolide (PLGA), polyesters, poly (orthoester), poly(phosphazine), poly (phosphate ester), polycaprolactones, or a combination thereof. A preferred polymeric carrier is PLGA. The release modifier is typically a long chain fatty alcohol, preferably comprising from 10 to 40 carbon atoms. Commonly used release modifiers include capryl alcohol, pelargonic alcohol, capric alcohol, lauryl alcohol, myristyl alcohol, cetyl alcohol, palmitoleyl alcohol, stearyl alcohol, isostearyl alcohol, elaidyl alcohol, oleyl alcohol, linoleyl alcohol, polyunsaturated elaidolinoleyl alcohol, polyunsaturated linolenyl alcohol, elaidolinolenyl alcohol, polyunsaturated ricinoleyl alcohol, arachidyl alcohol, behenyl alcohol, erucyl alcohol, lignoceryl alcohol, ceryl alcohol, montanyl alcohol, cluytyl alcohol, myricyl alcohol, melissyl alcohol, and geddyl alcohol.

Preferably, the non-antibody VEGF antagonist is incorporated into a microsphere-based sustained release composition. The microspheres are preferably prepared from PLGA. The amount of non-antibody VEGF antagonist incorporated in the microspheres and the release rate of the non-antibody VEGF antagonist can be controlled by varying the conditions used for preparing the microspheres.

Processes for producing such slow-release formulations are described in US 2005/0281861 and US 2008/0107694.

### Combination Therapy

According to the invention, patients receive both photodynamic therapy and a non-antibody VEGF antagonist. Administration of the non-antibody VEGF antagonist is performed before or after photodynamic therapy. Typically, administration of the non-antibody VEGF antagonist and photodynamic therapy will be performed on the same day (e.g. within 24 hours of one another). In this case, photodynamic therapy is generally performed first followed by administration of the non-antibody VEGF antagonist. In one embodiment, treatment with non-antibody antagonist is started up to 48 hours before photodynamic therapy. Alternatively, treatment with non-antibody VEGF antagonist is initiated at least 1 week, 2, weeks, 3 weeks, 4 weeks, 2 months, 3 months, 4 months, 5 months or 6 months before photodynamic therapy. The non-antibody VEGF antagonist may be administered every 4 weeks, every 6 weeks, or every 8 weeks. Treatment may be continued at the same interval or extended intervals after photodynamic therapy. Where the interval is extended, the period between administration of the non-antibody VEGF antagonist may increase by 50% or 100%. For example, if the initial interval was 4 weeks, the interval may be extended to 6 or 8 weeks. Preferably, administration of the non-antibody VEGF antagonist is once every eight weeks after every four weeks (monthly) for the first three months.

In some cases, a single injection of the non-antibody VEGF antagonist according to the invention may be sufficient to prevent recurrence of PCV. In other cases, three injections each one month apart are administered to the patient, while any subsequent injections are performed less frequently or on an as needed-basis. In certain cases, two or more injections spaced 6 weeks apart, preferably 8 weeks apart, more preferably 10 weeks apart may be required to improve visual acuity or halt disease progression. In other cases, three or more injections may be needed. In these cases, the time between injections should be at least 6 weeks, preferably 8 weeks, more preferably 10 weeks apart.

In another, preferred aspect of the invention, the non-antibody VEGF antagonist according to the invention is administered as needed. For example, after completion of the first treatment with photodynamic therapy and non-antibody VEGF antagonist, the treated eye may be re-evaluated by a combination of slit-lamp evaluation and biomicroscopic fundus examination with optical coherence tomography (OCT) and/or fluorescein fundus angiography (FFA). Functional changes (best corrected visual acuity) may also be assessed by Early Treatment of Diabetic Retinopathy Study charts. Re-evaluation may take place at 4 weeks, 6 weeks, 8 weeks, or 16 weeks after the first photodynamic therapy session. Subsequent follow-up visits may take place at 4 weeks, 6 weeks, 8 weeks, or 16 weeks after the first re-evaluation. A second, third or further administration of the non-antibody VEGF antagonist is performed only if examination of the eye reveals signs of disease activity such as non-complete polyp regression (e.g., partial regression or no change in polyp regression) and/or a loss of best-corrected visual acuity (e.g., a loss of >5 letters from best measured value). Preferably, any subsequent administration of a non-antibody VEGF antagonist is only given if OCT, clinical examination and FFA of the affected eye during re-evaluation reveals non-complete polyp regression or other signs of disease activity.

Generally, at least one non-antibody VEGF antagonist injection is administered. In some embodiments, three initial injections at monthly intervals are administered to initiate treatment, and further injections are given as needed if re-evaluation at subsequent follow-up visits reveals signs of disease activity.

Alternatively, non-antibody VEGF antagonist administration may be continuous, for example, if a PDS is used. The PDS may be implanted prior to photodynamic therapy. Alternatively, a single administration of non-antibody VEGF antagonist shortly before or after photodynamic therapy may be sufficient to achieve the desired effect. For example, a single dose of non-antibody VEGF antagonist may be given on the day of the photodynamic therapy.

Photodynamic therapy may be repeated as needed. Generally, it is not given more frequently than every 3 months. Photodynamic therapy may be repeated every 3 months. For example, additional photodynamic therapy may be given at least 3 months following a previous photodynamic therapy session if only partial regression or no change in polyp regression is observed during follow-up evaluations. For instance, if leakage is observed, e.g. as assessed by indocyanine green angiography (ICGA) or FAA, retreatment with photodynamic therapy is performed. Treatment may be continued with photodynamic therapy until there has been complete regression of polyps in the treated eye(s). Preferably, the need for additional photodynamic therapy is assessed using ICGA. Alternatively, photodynamic therapy may be repeated less frequently, in particular if the non-antibody VEGF antagonist treatment is continued after photodynamic therapy. For example, intervals between photodynamic therapy may be extended to every 4 months, every 5 months, every 6 months. In some cases, continued treatment with a non-antibody VEGF antagonist treatment prevents recurrence of PCV.

### Patient

The invention is useful for treating human patients with PCV. Diagnosis of PCV can be made by clinical examination in combination with OCT and FFA. To better determine choroidal involvement and to track polyp regression in response to treatment, the affected eye is typically also assessed by ICGA at baseline prior to initiation of treatment of photodynamic therapy and/or non-antibody VEGF antagonist therapy.

The patient can be any age, but will typically be an adult, and may be elderly (e.g. at least 65 years old). Often the patient will be of Asian descent (excluding Asians of Indian ancestry) or African-American, as such patients are more at risk for developing PCV.

The patient ideally does not have porphyria. Similarly, the patient ideally does not have severe hepatic impairment. Verteporfin is contraindicated in such patients.

Patients who receive verteporfin are photosensitive for 48 hours after infusion. During that period, they should avoid exposure of unprotected skin, eyes or other body organs to direct sunlight or bright indoor light such as tanning salons, bright halogen lighting, or high power lighting in surgery operating rooms or dental surgeries. If patients have to go outdoors in daylight during the first 48 hours after treatment they should protect their skin and eyes by wearing protective clothing and dark sunglasses.

### General The term “comprising” encompasses “including” as well as “consisting” e.g. a composition “comprising” X may consist exclusively of X or may include something additional e.g. X+Y.

The term “about” in relation to a numerical value x is optional and means, for example, x+10%.

### MODES FOR CARRYING OUT THE INVENTION

### Comparative Example 1

Sixty-one patients were randomized to three treatment groups: verteporfin photodynamic therapy, ranibizumab 0.5 mg, or the combination. Patients were administered with verteporfin photodynamic therapy/placebo and initiated with three consecutive monthly intravitreal ranibizumab/sham injections starting from day 1 (first photodynamic therapy session). Indocyanine green angiography was performed at baseline and at months 3, 4, 5, and 6. Fluorescein angiography was performed to assess the presence of leakage and to obtain fundus photographs at the same times. Changes in central retinal thickness were determined by optical coherence tomography. Treatment with ranibizumab/sham injections was repeated at months 3-5 on an as-needed basis.

The primary endpoint was the proportion of patients with indocyanine green angiography-assessed complete regression of polyps at month 6. Secondary endpoints included mean change in best corrected visual acuity at month 6 and safety.

At month 6, verteporfin combined with ranibizumab or alone was superior to ranibizumab monotherapy in achieving complete polyp regression (77.8% and 71.4% vs. 28.6%; P>0.01). Mean change±standard deviation in best corrected visual acuity (letters) was 10.9±10.9 (verteporfin photodynamic therapy+ranibizumab), 7.5±10.6 (verteporfin photodynamic therapy), and 9.2±12.4 (ranibizumab). All treatments were well tolerated over 6 months. There were no new safety findings with either drug used alone or in combination.

### Example

Male and female patients, 50 years and older, diagnosed with symptomatic macular Polypoidal Choroidal Vasculopathy (PCV), are randomized to two treatment groups: (1) aflibercept 2 mg in combination with sham photodynamic therapy, or (2) aflibercept 2 mg in combination with verteporfin photodynamic therapy.

Indocyanine green angiography is performed at baseline to determine patient suitability for inclusion. Patients are excluded who have undergone intravitreal or sub-tenon corticosteroids treatment in the study eye within 3 months prior to study entry; any treatment with intraocular anti Vascular Endothelial Growth Factor (anti-VEGF) agents in the study eye, or systemic treatment with anti-VEGF products within 3 months prior to study entry; prior macular laser treatment in the study eye including Photodynamic Therapy (PDT). Patients are also excluded if there is a history of allergy to fluorescein used in fluorescein angiography, iodine and/or indocyanine green; or history of allergy to aflibercept, verteporfin, or their excipients.

Patients are administered with intravitreal aflibercept injections according to the approved label. Verteporfin photodynamic therapy, when administered, is also given in accordance with the approved label. The primary outcome is the proportion of patients with a loss of less than 15-letters on BCVA scale at week 52.

It will be understood that the invention is described above by way of example only and modifications may be made whilst remaining within the scope and spirit of the invention.

1. A method for treating a patient having polypoidal choroidal vasculopathy, comprising administering a therapeutically effective combination of both (i) a photodynamic therapy and (ii) a non-antibody VEGF antagonist.

2. The method of claim 1, wherein the photodynamic therapy is verteporfin.

3. The method of claim 2, wherein the verteporfin is administered by intravenous infusion.

4. The method of claim 1, wherein the non-antibody VEGF antagonist is aflibercept.

5. The method of claim 4, wherein the aflibercept is administered via intravitreal injection.

6. The method of claim 5, wherein the aflibercept is administered at a dose of 2 mg.

7. The method of claim 1, wherein the patient receives more than one administration of antagonist.

8-12. (canceled)


SEQUENCE LISTING (3 sequences)

SEQ ID NO: 1 - protein, 431 aa, Artificial Sequence
  Aflibercept
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 552 aa, Artificial Sequence
  Conbercept
MVSYWDTGVLLCALLSCLLLTGSSSGGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRLPAKYLGYPPPEIKWYKNGIPLESNHTIKAGHVLTIMEVSERDTGNYTVILTNPISKEKQSHVVSLVVYVPPGPGDKTHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 3 - protein, 126 aa, Artificial Sequence
  DARPin MP0112
GSDLGKKLLEAARAGQDDEVRILMANGADVNTADSTGWTPLHLAVPWGHLEIVEVLLKYGADVNAKDFQGWTPLHLAAAIGHQEIVEVLLKNGADVNAQDKFGKTAFDISIDNGNEDLAEILQKAA